(19) United States
(12) Patent Application Publication
(10) Pub. No.: US 2018/0000975 A1
(43) Pub. Date: Jan. 4, 2018

(54) DEVICE FOR CLEANING AND STERILIZING A FILLING VALVE OF A BEVERAGE FILLING SYSTEM FOR FILLING A CONTAINER WITH A FILLING PRODUCT

(71) Applicant: KRONES AG, Neutraubling (DE)

(72) Inventor: Erwin Knieling, Neutraubling (DE)

(21) Appl. No.: 15/545,613

(22) PCT Filed: Jan. 22, 2016
(86) PCT No.: PCT/EP2016/051335

§ 371 Date: Jul. 21, 2017

(30) Foreign Application Priority Data
Jan. 22, 2015 (DE) 10 2015 100 893.9

(51) Int. Cl. A61L 2/07; A61L 2/26; F16K 31/00

(52) U.S. Cl. CPC A61L 2/07; F16K 31/002; A61L 2/26; A61L 2202/15; A61L 2202/17

(57) ABSTRACT

The present invention relates to a device and a method for cleaning and sterilizing a filling valve of a beverage filling system for filling a container with a filling product. The device includes a cap for closing a filling product outlet of the filling valve during the cleaning and sterilization, and a valve for opening and closing the through-opening. The cap has a through-opening for discharging sterilization medium during the sterilization. The valve includes a shape-memory material for switching the valve between an open and closed position at a predetermined temperature.

### CROSS REFERENCE TO RELATED APPLICATIONS

This application is a national stage of International Application No. PCT/EP2016/051335, filed Jan. 22, 2016, which claims priority from German Patent Application No. 10 2015 100 893.9 filed on Jan. 22, 2015 in the German Patent and Trademark Office, the disclosures of which are incorporated herein by reference in their entirety.

### BACKGROUND

### Technical Field

The present invention relates to a device and a method for cleaning and sterilizing a filling valve of a beverage filling system for filling a container with a filling product, comprising a cap for closing the filling product outlet of the filling valve during the cleaning and sterilization, wherein the cap has a through-opening for discharging sterilization medium during the sterilization, and a valve for opening and closing the through-opening.

Related Art

Filling operations of a filler in a beverage filling system are regularly interrupted by a cleaning and sterilization phase. This is performed in order to ensure regular cleaning and sterilization, for example every 16 hours. In addition, the cleaning and sterilization procedure can be carried out when the product is changed, in order to prepare the production process for the next product.

In order to clean a filling valve, it is known to close the product outlet aperture with a rinsing cap, in order reliably to rinse, with a recirculating cleaning medium, the portion of the filling valve that comes into contact with the product. The rinsing cap completely closes the product outlet aperture, so that no cleaning medium is lost or has an adverse effect on the environment.

A cap is also provided in front of the product outlet aperture during the sterilization operation, in order that sterilization can be carried out with recirculating saturated steam. In the process, condensate forms in the interior of the filler, with the result that it is necessary to provide a discharge outlet at the lowest extremities of the filler, through which the condensate can drain. In order to maintain the temperature of the steam, it is further arranged that a portion of the steam flows through the discharge outlet, thus achieving a constant transport of fresh steam through the interior of the filler. The discharge outlet is normally provided in the form of a drilled hole in the steam cap that closes the product outlet aperture during the sterilization operation.

Accordingly, two different caps are usually provided for the cleaning operation and the sterilization operation, namely a closed rinsing cap for the cleaning and a steam cap with a drilled hole for the sterilization. Sufficient space must therefore be provided in the filler to hold both caps in readiness. In addition, each cap requires its own pivoting device, by means of which the cap can be positioned beneath the filling valve. Furthermore, the changeover of the caps between the cleaning operation and the sterilization operation contributes to a prolongation of the interruption of filler operation.

### SUMMARY

Proceeding from the known state of the art, the present invention proposes an improved device and an improved method for cleaning and sterilizing a filling valve of a beverage filling system for filling a container with a filling product.

Accordingly, in one embodiment, a device for cleaning and sterilizing a filling valve of a beverage filling system for filling a container with a filling product is proposed, which includes a cap for closing a filling product outlet of the filling valve during the cleaning and sterilization, wherein the cap has a through-opening for discharging sterilization medium during the sterilization, and a valve for opening and closing the through-opening. According to one embodiment of the invention, the valve includes a shape-memory material, for example a shape-memory alloy, for switching the valve between an open and a closed position at a predetermined temperature.

By this means it is possible to carry out both the cleaning of the filling valve and the sterilization of the filling valve with the same cap. During a cleaning operation, the filling valve is rinsed with a cleaning medium, for example water, lye, acid or a similar material. In this case, the cap disposed on the filling valve prevents the cleaning medium from leaving a cleaning circulation in which all parts of a filler system that come into contact with the product, in particular the filling element, are rinsed. During the cleaning operation, a through-opening in the cap would lead to the cleaning medium, which is mixed with filling product residues, escaping from the cap and contaminating surrounding areas of the system.

During the sterilization operation, in which a sterilization medium, for example in the form of saturated steam, flows through the filling element, in particular the filling valve, it is necessary to have a through-opening in the cap which enables the condensate that forms in the filling element during sterilization to drain out. In addition, by means of the drainage of a portion of the sterilization medium from the thorough-opening, the process of sterilization can be monitored using suitable sensors.

Because a switchable through-opening is provided, the proposed cap is suitable for both the cleaning operation and the sterilization operation. Because only one cap is required, both material and installation space can be saved. Furthermore, it is no longer necessary to exchange the cap between the cleaning operation and the sterilization operation, which shortens the time required to carry out the cleaning and sterilization of the filling valve.

Because, due to its shape-memory material, the valve can be switched depending on a predetermined temperature, it is not necessary to switch the valve actively by means of a manual intervention or by control of an actuator.

Shape-memory materials in the form of shape-memory alloys can have two different crystal structures, depending on the temperature. Thus the shape of a shape-memory alloy can be changed by selecting the temperature. Due to the so-called two-way effect, a shape-memory alloy can have a first shape at a first temperature and a second shape at a second temperature. It can hence be used as a thermally operated actuator.

In the proposed device, the temperature-dependent change of the shape-memory material from a first shape to a second shape, and vice-versa, effects the switching of the valve between the open and closed positions. For example, the properties of the shape-memory material can be such that in the range of temperatures that occurs during the cleaning operation, it holds the valve in a closed position, and in the range of temperatures that occur during the sterilization operation, it holds the valve in an open position.

In various embodiments, the properties of the shape-memory material are such that the transition from a first shape of the shape-memory material to a second shape of the shape-memory material takes place in a temperature range that lies between the temperature range for the cleaning operation and the temperature range for the sterilization operation. By this means it can be ensured that, within the respective temperature ranges of the cleaning operation and the sterilization operation, the shape of the shape-memory alloy does not change even when the temperature fluctuates, and thus the desired valve position in each case is maintained.

Furthermore, because the two-way effect is utilized to switch the valve between the open position and the closed position, it is possible to dispense with electrical actuation of the valve. Accordingly, no electrical cabling or other supply line for working media is needed to connect the valve with a control or regulating unit. In addition, no direct energy supply is needed to switch the valve between the open position and the closed position. This has advantageous effects on the operating costs and the reliability of the device, and in particular of the valve.

In several embodiments, the valve includes a positioning element for opening and closing the valve, wherein the positioning element includes the shape-memory material. Because the positioning element is formed from the shape-memory material, the positioning element has the properties of a temperature-dependent actuator. In accordance with the two-way effect, the positioning element can “remember” two shapes, such that, at a predetermined higher temperature and at a predetermined lower temperature, it can adopt the applicable shape. In this manner the shape-memory material confers on the positioning element the property that in the first shape, it closes the valve, and in the second shape, it leaves the valve open.

Because the cleaning of the filling valve normally takes place at lower temperatures than the sterilization of the filling valve, it is advantageous to design the device such that the positioning element closes the valve in the region of a predetermined lower temperature, and in the region of a predetermined higher temperature, it enables an open position of the valve. By this means, it is ensured that the through-opening of the cap is kept closed during cleaning, so that the cleaning medium does not escape from the cap, and during sterilization, the through-opening of the cap is open, so that condensate which is formed when steam is applied to the filling valve can be discharged via the through-opening.

In some embodiments, the valve includes a closing part and a valve seat, wherein, in a closed position of the valve, the closing part is arranged in a sealing manner in the valve seat, and in an open position of the valve, the closing part is raised out of the valve seat, wherein the positioning element can be brought into contact with the closing part in order to switch the valve.

Accordingly, movement of the closing part relative to the valve seat can be enabled by means of the positioning element. For example, by means of changing the length of the positioning element, the closing part can be arranged in the valve seat or released from it. The positioning element thus makes it possible for the closing part to be arranged in the valve seat at a predetermined temperature for the cleaning of the filling valve, and for the closing part to be released from, or not arranged in, the valve seat at a predetermined temperature for the sterilization of the filling valve.

In several embodiments, the positioning element is a spring, for example, a helical spring, which is formed from the shape-memory material.

By this means, at a predetermined first temperature the closing part can be pressed into the valve seat in a sealing manner, so that the through-opening of the cap is closed and cleaning of the filling valve can be carried out. The spring can have the property that, due to the two-way effect of the shape-memory material, it has a shortened shape at a predetermined second temperature. When the spring has its shortened shape, the closing part is not pressed into the valve seat, so that the valve is in the open position. As a result, the through-opening of the cap is in an open position, so that sterilization of the filling valve can be carried out.

In certain embodiments, a pivoting device, or pivoting and lifting device, is provided, by means of which the cap can be positioned on the filling product outlet of the filling valve.

The cap can thus be automatically arranged on the filling product outlet of the filling valve in order to carry out cleaning and/or sterilization of the filling valve, without the need for manual intervention. The lifting device provides a lifting movement, by means of which the cap is placed on, or can be released from, the filling product outlet of the filling valve. The lifting device can also be provided in the form of a pneumatic cylinder, which is capable of performing lifting movements by applying and relieving air pressure, in order to arrange the cap on the filling valve.

In addition, the device can have a pivoting device, by means of which the cap can be pivoted to beneath the filling product outlet of the filling valve, in order subsequently to be arranged on the filling product outlet of the filling valve by means of the lifting device.

The cap can further be disposed on a lifting plate, which is provided for accommodating and transporting a container that is to be filled. By this means, use can be made of the lifting and pivoting device of the lifting plate in order to arrange the cap on the filling valve.

In some embodiments, the shape-memory material includes a shape-memory alloy such as nitinol, nickel-titanium, nickel-titanium-copper, copper-zinc, copper-zinc-aluminum, copper-aluminum-nickel, iron-nickel-aluminum, iron-manganese-silicon and/or zinc-gold-copper.

Such alloys are capable of existing in two different crystal structures, and can, due to the two-way effect, “remember” two shapes. It is thus possible to use these alloys to form shape-memory alloys, by means of which the valve can be switched depending on the temperature.

Accordingly, a method for cleaning and sterilizing a filling valve of a beverage filling system for filling a container with a filling product is proposed, which includes use of a device in accordance with the aspects described above, with the steps of arranging the cap on the filling product outlet of the filling valve, cleaning the filling valve in the cleaning operation at a first temperature, sterilizing the filling valve in the sterilizing operation at a second temperature, wherein at the second temperature the valve switches to the open position, and removing the device from the filling valve.

With the aid of this method, the advantages that have been described above with regard to the device are also achieved.

In several embodiments, the first temperature in the filling valve, and in particular in the device, during the cleaning operation causes the shape-memory material to keep the valve closed.

By this means, it can be ensured that at the temperatures occurring during the cleaning operation, the through-opening of the cap remains closed. This prevents cleaning medium from escaping from the through-opening of the cap during the cleaning operation. The first temperature can be, for example, below 85° C.

In some embodiments, the second temperature in the filling valve, and in particular in the device, during the sterilization operation causes the shape-memory material to keep the valve open.

This makes it possible during the sterilization, for example using steam, of the filling valve, for condensate which forms in the filling valve to be discharged via the through-opening of the cap. The second temperature can be, for example, greater than 105° C.

### BRIEF DESCRIPTION OF THE FIGURES

Further embodiments and aspects of the present invention are more fully explained by the description below of the figures.

FIG. 1 is a schematic sectional view of a device for cleaning and sterilizing a filling valve, wherein a positioning element which is disposed in a through-opening holds a valve in a closed position,

FIG. 2 is a schematic sectional view of the device from FIG. 1, wherein the positioning element holds the valve in an open position,

FIG. 3 is a schematic sectional view of a further device for cleaning and sterilizing a filling valve, wherein a positioning element which is disposed perpendicular to a through-opening holds a valve in a closed position, and

FIG. 4 is a schematic sectional view of the device from FIG. 3, wherein the positioning element holds the valve in an open position.

### DETAILED DESCRIPTION

Examples of embodiments are described below with the aid of the figures. In the figures, elements which are identical or similar, or have identical effects, are designated with identical reference signs. In order to avoid redundancy, repeated description of these elements is in part dispensed with in the description below.

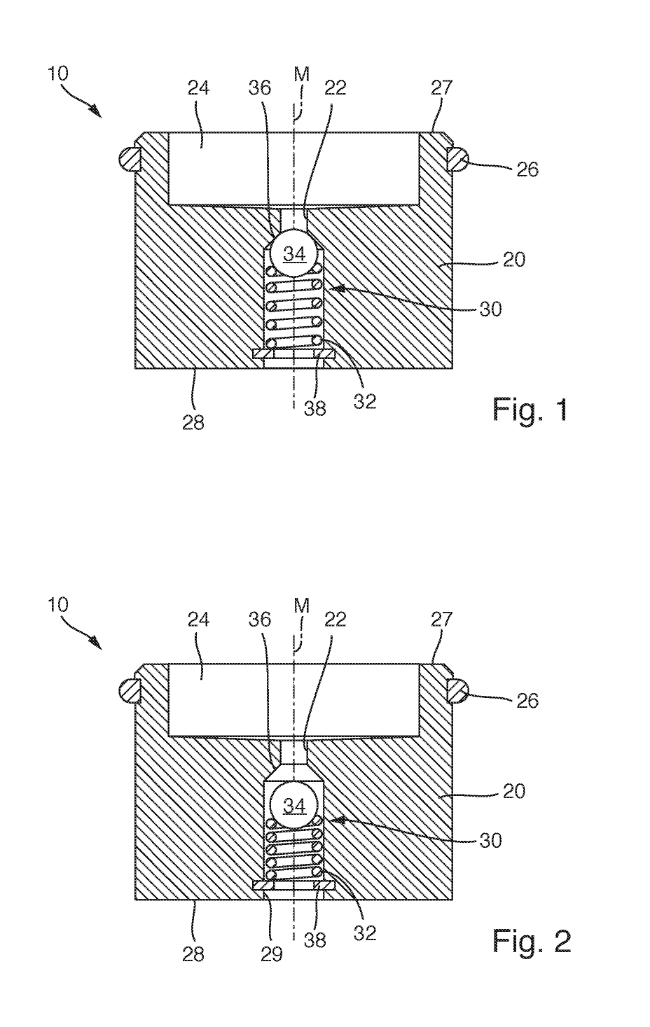

FIG. 1 shows a device 10 by means of which a filling valve of a beverage filling system can be closed for cleaning and sterilization. The device 10 includes a cap 20, which can be arranged on the product dispensing aperture of the filling valve (which is not shown here) in order to close the product dispensing aperture. The cap 20 is cylindrical, and has, in an upper region of an outer circumferential surface, a ring seal 26. In order to seal the filling valve, the ring seal 26 is in contact with a surface of the dispensing aperture of the filling valve, in particular with the conical wall of a centering bell.

The cap 20 has on an upper side 27 a drilled hole 24. The drilled hole 24 serves to accommodate elements of the filling valve, such as for example the end of a gas return pipe, in a contactless manner in the interior of the cap 20, and to redirect the cleaning medium during the clean-in-place (CIP) cleaning. There is a gap between the elements of the filling valve and the surface of the drilled hole 24 when installed, which for example enables fluid to flow around the elements of the filling valve during cleaning operation. The drilled hole 24 is disposed concentrically to the middle axis M of the cap 20.

In the base of the drilled hole 24, concentrically to the middle axis M of the cap 20, a through-opening 22 is disposed, which extends to an underside 28 of the cap 20. In the through-opening 22 a valve 30 is disposed, by means of which the through-opening 22 can be opened and closed, in order to switch between a cleaning operation and a sterilization operation of the device 10.

The valve 30 has a valve seat 36, in which a closing part 34 in the form of a stainless steel sphere is accommodated. In this state, the through-opening 22 is closed, so that the device 10 is in a cleaning position. The closing part 34 is pretensioned against the valve seat 36 by means of a positioning element 32. The positioning element 32 is designed in the form of a spring, which extends, in the shape of a helix, concentrically to the middle axis M of the device 10. In the lower region of the through-opening 22, a retaining ring 38 is disposed, against which the positioning element 32 is braced.

The positioning element 32 is formed from a shape-memory material in the form of a shape-memory alloy including nickel and titanium, for example nitinol. The positioning element 32 is accordingly capable of changing its shape, in particular its length, depending on the temperature. The positioning element 32 thereby has the property that during a cleaning operation in which a first temperature prevails, it has the shape shown in FIG. 1, by which the closing part 34 is pretensioned against the valve seat 36. The valve 30 is accordingly closed as a result of the temperature which prevails during the cleaning operation. For example, the cleaning operation can be designed such that a temperature of 80° C. is not exceeded. The shape-memory alloy of the positioning element 32 thus has the property that, up to a temperature of 80° C., it does not change the shape shown in FIG. 1. Alternatively, the properties of the shape-memory alloy can be such that the positioning element 32 maintains its first shape up to a temperature that is lower or higher than 80° C.

The shape-memory alloy of the positioning element 32 has the further property that it adopts the shape shown in FIG. 2 at the temperature prevailing during the sterilization operation. In the device 10 that is shown in FIG. 2, the positioning element 32, i.e. the spring, is shortened, because the shape-memory alloy has a different crystal structure at the second temperature, which prevails during the sterilization operation. The shape of the positioning element 32 that is shown in FIG. 2 exists at and above a sterilization temperature of 105° C. Alternatively, the second shape of the positioning element 32 can also be adopted at a lower or higher temperature. Due to the shortened positioning element 32 that is shown in FIG. 2, the closing part 34 is released from the valve seat 36, with the result that the valve 30 is in an open position. The through-opening 22 through the cap 20 is thus open, so that condensate that forms in the filling valve during the sterilization operation can flow out through the through-opening 22. In this case, a volume flow of the condensate flows between the inner circumferential surface of the through-opening 22 and the closing part 34, through the positioning element 32, and through the retaining ring 38, and finally flows out of an aperture 29 in the underside 28 of the cap 20.

When the device 10, and in particular the positioning element 32, cools again after the sterilization operation, the positioning element 32 again adopts the first shape shown in FIG. 1, by which the closing part 34 is pretensioned against the valve seat 36.

The cap 20 is manufactured from stainless steel. Alternatively, the cap 20 can be manufactured from plastic.

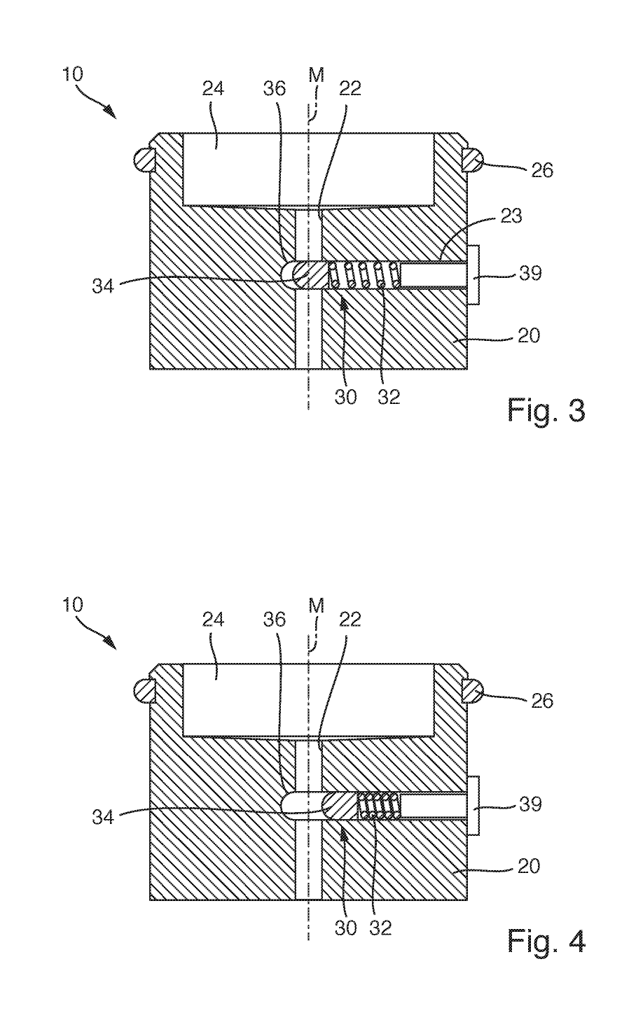

FIG. 3 shows a device 10 which differs from the devices 10 shown in FIGS. 1 and 2 in that the positioning element 32 is not disposed in the through-opening 22. In the device shown in FIG. 3, the through-opening 22 extends from the base of the drilled hole 24, concentrically to the middle axis M of the device 10, to the underside 28 of the cap 20. The cap 20 has a drilled hole 23 which extends perpendicular to, and crosses, the through-opening 22. At one end, the drilled hole 23 has a valve seat 36. The closing part 34 is disposed in the drilled hole 23. In the device 10 that is shown in FIG. 3, the closing part 34 is arranged in the valve seat 36. The closing part 34 thus closes the through-opening 22 that extends perpendicular to the drilled hole 23, so that the device 10 is in the cleaning position. The closing part 34 is pretensioned by the positioning element 32 against the valve seat 36. The positioning element 32 is braced against a bolt 39, which is accommodated in the drilled hole 23. The positioning element 32 is designed in the shape of a spring, whose ends are fixed to the closing part 34 and the bolt 39. The positioning element 32 that is shown in FIG. 3 is manufactured from a shape-memory alloy which comprises nickel, titanium and copper. The positioning element 32 shown in FIG. 3 has the shape of the positioning element 32 during the cleaning operation of the filling valve.

During the sterilization operation, the positioning element 32 adopts the shape that is shown in FIG. 4. The shape of the positioning element 32 that is shown in FIG. 4 is shorter in comparison with the shape of the positioning element 32 that is shown in FIG. 3. In the position of the valve 30 that is shown in FIG. 4, the closing part 34 is not accommodated in the valve seat 36. Hence the through-opening 22 of the cap 20 is open, so that condensate that arises during the sterilization operation can flow out through the through-opening 22.

When the temperature falls after the sterilization operation has ended, the shape-memory alloy in the positioning element 32 causes the positioning element to readopt the shape shown in FIG. 3. The positioning element, which is designed in the shape of a spring, accordingly pushes the closing part 34 back into the valve seat 36. By this means the valve 30 is closed.

To the extent applicable, all individual features described in the individual example embodiments can be combined with each other and/or exchanged, without departing from the field of the invention

1-9. (canceled)

10. A device for cleaning and sterilizing a filling valve of a beverage filling system, comprising:
a cap configured to close a filling product outlet of the filling valve during cleaning and sterilization, wherein the cap comprises:
a through-opening configured to discharge sterilization medium during the sterilization, and
a valve comprising a shape-memory material that is configured to switch the valve between an open position and a closed position at a predetermined temperature.

11. The device of claim 10, wherein the valve further comprises a positioning element configured to open and close the valve.

12. The device of claim 11, wherein the positioning element comprises a spring.

13. The device of claim 12, wherein the spring comprises a helical spring.

14. The device of claim 12, wherein the positioning element comprises a shape-memory material.

15. The device of claim 11, wherein the valve further comprises a closing part and a valve seat, and wherein in a closed position of the valve, the closing part is arranged in a sealing manner in the valve seat, and in an open position of the valve, the valve is raised out of the valve seat.

16. The device of claim 15, wherein the positioning element is configured to be brought into contact with the closing part to switch the valve in between the open position and the closed position.

17. The device of claim 10, wherein the shape-memory material comprises a shape-memory alloy.

18. The device of claim 17, wherein the shape-memory alloy comprises nitinol, nickel-titanium, nickel-titanium-copper, copper-zinc, copper-zinc-aluminum, copper-aluminum-nickel, iron-nickel-aluminum, iron-manganese-silicon, zinc-gold-copper, or combinations thereof.

19. The device of claim 10, further comprising an opening disposed on an upper side of the cap that is concentric with the cap.

20. The device of claim 10, further comprising an opening that extends perpendicular to and crosses the through-opening.

21. A device for cleaning and sterilizing a filling valve of a beverage filling system, comprising:
a cap configured to close a filling product outlet of the filling valve during cleaning and sterilization, wherein the cap comprises:
a through-opening configured to discharge sterilization medium during the sterilization; and
a valve comprising:
a positioning element configured to open and close the valve, wherein the positioning element comprises a shape-memory alloy,
a closing part, and
a valve seat,
wherein in a closed position of the valve, the closing part is arranged in a sealing manner in the valve seat and in an open position of the valve, the valve is raised out of the valve seat.

22. The device of claim 21, wherein the positioning element is configured to be brought into contact with the closing part to switch the valve in between the open position and the closed position.

23. The device of claim 21, wherein the shape-memory alloy comprises nitinol, nickel-titanium, nickel-titanium-copper, copper-zinc, copper-zinc-aluminum, copper-aluminum-nickel, iron-nickel-aluminum, iron-manganese-silicon, zinc-gold-copper, or combinations thereof.

24. A method for cleaning and sterilizing a filling valve of a beverage filling system, comprising:
arranging a cap on a filling product outlet of the filling valve, wherein the cap comprises a through-opening configured to discharge sterilization medium during the sterilization, and a valve comprising a shape-memory material that is configured to switch the valve between an open position and a closed position at a predetermined temperature;
cleaning the filling valve in a cleaning operation at a first temperature;
sterilizing the filling valve in a sterilization operation at a second temperature, wherein the valve switches to the open position at the second temperature; and
removing the cap from the filling valve.

25. The method of claim 24, wherein the first temperature causes the shape-memory material to keep the valve closed.

26. The method of claim 24, further comprising providing a pivoting device, a lifting device, or both, to arrange the cap on the filling product outlet.

27. The method of claim 24, further comprising pivoting the cap beneath the filling product outlet.

28. The method of claim 24, wherein the first temperature is below 85° C.

29. The method of claim 24, wherein the second temperature is greater than 105° C.
